(19) United States
(12) Patent Application Publication
(10) Pub. No.: US 2026/0290576 A1
(43) Pub. Date: Sep. 24, 2026

(54) Method and System for Feedback Data Collection

(71) Applicant: Sydekick Robotics Pty Ltd, Kelvin Grove (AU)

(72) Inventor: David Rodney Pershouse, Bray Park (AU)

(21) Appl. No.: 19/466,305

(22) Filed: Jan. 31, 2026

(30) Foreign Application Priority Data
Jan. 31, 2025 (AU) 2025900257

(51) Int. Cl. G16H 40/67; B25J 9/16; G05B 13/02; G06N 3/008; G06N 3/096

(52) U.S. Cl. CPC G16H 40/67; B25J 9/1689; G05B 13/027; G06N 3/008; G06N 3/096

(57) ABSTRACT

A teleoperation device for use in collecting data for fine-tuning a machine learning model to control a robotic device, the teleoperation device comprising a morphology matching kinematic chain structure corresponding to a kinematic chain structure of the robotic device, wherein each joint and link of the robotic device has a corresponding joint and link on the teleoperation device, actuators configured to enable movement of the teleoperation device joints under autonomous control of a machine learning model or under operator input, control circuitry configured to synchronise movement of the teleoperation device with movement of the robotic device such that movements of one device are mirrored by the other and a data interface configured to transmit operator input data and corrective behaviours to a server for aggregation into a training dataset associated with the machine learning model.

### FIELD OF THE INVENTION

The present disclosure relates to robotics, machine learning, and teleoperation systems. Specifically, the present disclosure relates to methods and systems for collecting interactive demonstration data for use in robotics and machine learning. More specifically, the disclosure relates to methods and systems for interactive human-in-the-loop data collection, dataset augmentation, and iterative refinement of robot learning control policies.

### BACKGROUND TO THE INVENTION

In recent years, data-driven methods such as imitation learning and reinforcement learning have gained prominence in robotics, offering the ability to capture complex behaviours that were previously unattainable with classical robotics techniques. These methods rely on large, high-quality datasets to train robot policies, enabling them to perform intricate tasks autonomously. However, the performance of these systems is highly sensitive to the quality and diversity of the data used for training.

Imitation learning, for example, involves training robots by mimicking demonstration data collected from human operators. While effective, this approach often struggles when the robot encounters situations outside the training distribution-commonly referred to as being “out of distribution.” In such cases, the robot's learned policy may fail to generalize, leading to errors or suboptimal behaviour. Addressing these edge cases is critical to ensuring robust and reliable performance.

Traditional data collection methods lack the flexibility to incorporate human corrections during policy execution, limiting the ability to refine policies in real time. Without an effective way to guide robots back into distribution, errors persist and compound during deployment, resulting in significant performance degradation.

To address these limitations, there is a need for a solution to bridge the gap between actual policy performance and the desired performance required to enable robotic devices to learn to recover from errors, learn from edge cases and achieve levels of performance and generalisation that are essential for real-world deployment.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

In accordance with an aspect of the invention there is provided a method for collecting data for fine-tuning a machine learning model to control a robotic device, the method comprising the steps of:
receiving a request for initiating an action to be performed by a robotic device;
processing the request to identify a machine learning model associated with the requested action;
providing access to the machine learning model to a controller of one or both of the robotic device a teleoperation device and instructing the controller of the relevant device to execute the model, wherein the teleoperation device includes a morphology matching kinematic chain structure corresponding to a kinematic chain structure of the robotic device, wherein each join and link of the robotic device has a corresponding joint and link on the teleoperation device;
receiving feedback data from at least one of the controllers, the feedback data including data relating to a human-based input provided via the teleoperation device; and
processing the feedback data to update the machine learning model, wherein processing the feedback data includes:
   aggregating a training dataset associated with the machine learning model with the feedback data; and
   re-training the machine learning model using the aggregated training dataset.

In a preferred embodiment the method is being conducted at a server in a system for collecting data for training a machine learning model.

The step of processing the request to extract data therefrom and identify a machine learning model may include extracting a user identifier and an action identifier from the action request. The user identifier may be associated with a user platform and/or user device and the action identifier may be associated with one or more machine learning models. The method may further include determining whether a user associated with the user identifier has access to the one or more machine learning models associated with the action identifier.

In a preferred embodiment the feedback data includes pre-processed data logs based on data captured by a sensor array of the robotic device and/or a sensor array of the teleoperation device. In some embodiments the feedback data may also include pre-processed data logs based on data captured by a user device.

The teleoperation device may be configured to have a corresponding kinematic structure to a kinematic structure of the robotic device so that the teleoperation device may mirror joint actions that may be performed by the robotic device. The teleoperation device may be configured to enable an operator to intervene during operation of the robotic device and/or teleoperation device to provide corrective behaviour captured that may be collected as feedback data.

In some embodiments the teleoperation device and the robotic device may be a single unified DEVICE.

The method may include determining a performance score of the model executed by the controller. In a preferred embodiment, determining the performance score includes processing the feedback data to identify data logs associated with actions performed by the robotic device; matching the actions associated with the data logs with a predefined success criteria to determine a ratio of a number of successful actions performed against a number of actions attempted; and determining a performance score based on the ratio. In another embodiment this may be a success criterion based on how many times the policy succeeds to solve the task when run on set of test environments. This could be in either the real world or in a simulation environment.

The method may include storing the machine learning model as a skill if the performance score is greater than a pre-determined threshold.

Providing access to the machine learning model to a controller of the robotic device or the teleoperation device and instructing the controller to execute the model may include transmitting the model data to the controller for processing and execution by the controller, or transmitting a pointer to the controller to provide the controller access to records in a model repository stored and maintained by the server.

In accordance with a further aspect of the invention there is provided a system for collecting data for fine-tuning a machine learning model to control a robotic device, the system comprising:
a memory for storing computer-readable program code and a processor for executing the computer-readable program code;
a request receiving component configured to receive a request for initiating an action to be performed by a robotic device;
a request processing component configured to process the request to identify a machine learning model associated with the requested action;
a machine learning component configured to provide access to the machine learning model to a controller of one or both of the robotic device and a teleoperation device and instructing the controller to execute the model;
a feedback receiving component configured to receive feedback data from at least one of the controllers, the feedback data including data relating to a human-based input provided via the teleoperation device; and
a feedback processing component configured to process the feedback data to update the machine learning model, wherein processing the feedback data includes:
   aggregating a training dataset associated with the machine learning model with the feedback data; and
   re-training the machine learning model using the aggregated training dataset.

In accordance with an even further aspect of the invention there is provided a system for collecting interactive feedback data for fine-tuning a machine learning model to control a robotic device, comprising:
a sever configured to store one or more machine learning models;
a robotic device configured to execute an action associated with the one or more machine learning models; and
a teleoperation device configured to, at least partially, be controlled by an operator via operator inputs,
wherein at least one of the teleoperation device or the robotic device is configured to mirror movements of the other device during execution of the one or more machine learning models,
wherein at least one of the teleoperation device or the robotic device includes a sensor array to record feedback data associated with the operator inputs during execution of the one or more machine learning models, and

wherein the server is in network communication with at least one of the robotic device and the teleoperation device and configured to process the captured feedback data and aggregate a training dataset associated with the machine learning model with the feedback data.

The disclosure therefore provides a method for collecting additional data for fine-tuning a machine learning model to control a robotic device. The method may comprise the following steps/series of steps:

Selecting a skill/policy from a skill library of previously trained skills for a wide range of different tasks. Each of these skills have been previously trained using robot learning methods such as imitation learning. These skills could be imperfect behaviours for a task or behaviours that only satisfy part of a larger task.

Once a skill is selected it is loaded onto a processing unit that can utilise this skill to control a robotic device.

The skill can take in as input state information which can include images of the robotic device's surroundings and the robotic device's state including joint and gripper information and then processes this information to generate a sequence of actions which correspond to the joint movements that the robotic device can take to solve the given task.

As these movements or behaviours may not be perfect to solve the desired task, there is a need to align these behaviours to solve the new given task selected by the user.

This is achieved with the aid of a morphology matching teleoperation device that can be used to teleoperate the robotic device. The teleoperation device may be configured to match the same kinematic chain structure as that of the robotic device that is to be controlled. Every joint and link on the robotic device may have a corresponding and, preferably, scaled down version on the morphology matching teleoperation device. As any joint on this teleoperation device moves, the movements may be mirrored on the actual robotic device. A key component to this teleoperation device is that every joint on this device can also be actuated and controlled similar to an actual robotic device.

The Inventors propose a human-in-the-loop, on-policy data collection method using these devices (robotic device and teleoperation device) to gather finetuning data to augment the learned behaviours of pretrained skills/policies.

By ensuring that the movements of the robotic device and the morphology matching teleoperation device are mirrored in both directions-if the robotic device moves the teleoperation device moves and vice versa, the actions of the pretrained policy can be directly applied to one of the devices.

This pretrained skill/policy then produces actions based on the input state provided to it and can be executed on the morphology matching teleoperation device or robotic device. As either of the devices moves based on the action commands stipulated by the pretrained skill/policy the other device moves synchronously to mirror the movements.

As the actual robotic device follows these movements to undertake a given task it may act erroneously or deviate from the desired behaviours expected by an operator, in control of the teleoperation device. In this case, the operator can switch the teleoperation device from autonomous mode to teleoperation mode where the operator can directly move the teleoperation device manually. As the teleoperation device and robotic device are still in-sync the operator's movements on the teleoperation device can be used to guide the actual robotic device to correct its behaviours in order to correctly complete the given task.

This mirrored movement/correction may happen seamlessly, in real-time, and the robotic device's operation does not stop but simply switches from being driven by a learned policy to being driven by the operator guided movements. The complete corrected trajectory consisting of the stream of states and actions taken by the robotic device to successfully complete the task are all recorded and stored to be uploaded and added to the original dataset used to train the skill. This augmented dataset can then be used to train a refined policy using imitation learning and the resulting new skill can be added to the skill library.

An embodiment of the invention will now be described, by way of example only, with reference to the accompanying drawings.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is a schematic diagram which illustrates an example embodiment of a system for collecting data for training a machine learning model according to aspects of the present disclosure;

FIGS. 2A and 2B are example embodiments of a teleoperation device according to aspects of the disclosure;

FIG. 2C shows example embodiments of teleoperation devices and their corresponding robotic devices according to aspects of the present disclosure;

FIG. 3 is a swim-lane flow diagram which shows example steps being performed by components of the system in a method for collecting data to train a machine learning model according to aspects of the present disclosure;

FIG. 4 is a flow diagram illustrating an example embodiment of a method for determining a model performance score;

FIG. 5 is a high-level block diagram which illustrates an exemplary server according to aspects of the present disclosure;

FIG. 6 is a high-level block diagram which illustrates an exemplary controller according to aspects of the present disclosure;

FIGS. 7A and 7B are example embodiments of a teleoperation device according to aspects of the disclosure;

FIGS. 8A and 8B are example embodiments of unified teleoperation and robotic devices;

FIGS. 9A to 9D are example embodiments of morphology matching teleoperation devices for controlling a robotic device; and,

FIG. 10 is a flow diagram illustrating an example embodiment for system flow architecture for iterative policy refinement.

### DETAILED DESCRIPTION WITH REFERENCE TO THE DRAWINGS

The below description is provided to facilitate an understanding of the invention described with reference to the accompanying Figures.

The method and system discussed herein is configured for refining and augmenting robot learning policies by enabling human-in-the-loop corrections during both autonomous and teleoperated control of a robotic device. The system includes a morphology-matching teleoperation device configured to mirror the kinematic structure of the robotic device associated therewith. The morphology-matching teleoperation device is configured to facilitate seamless, real-time human intervention to correct the behaviour of the robotic device, in use, thereby facilitating the collection of augmented data trajectories that are used for iterative machine learning policy refinement.

In a preferred embodiment of the system the morphology-matching teleoperation device is a teleoperation mechanism configured to closely replicate the kinematic chain of a target robotic system, which in this case is a robotic device, discussed in more detail below. In a preferred embodiment the morphology-matching teleoperation device includes one or more rotary encoders and actuators. The teleoperation device may include one or more portions including joints (for example articulated robotic arms) and each joint of the teleoperation device may include a rotary encoder arranged to measure joint positions and actuators arranged to enable motor-driven movements of the joints of the teleoperation device. The teleoperation device may further be configured to support both passive and active control modes. In the passive mode, a human operator of the device may directly drive the joints of the teleoperation device while the actuators move freely. In other words, the actuators may be configured to enable free, low friction movement of the joints when the teleoperation device is in the passive mode. Conversely, in the active mode, one or more torque sensors provided on the teleoperation device detect human input forces, and a low-level impedance-based controller applies velocity or torque commands to the actuators.

In some embodiments, the teleoperation device may be configured to mirror the movements of the robot device, discussed below, during autonomous execution. In further embodiments, the teleoperation device may be configured to drive the robotic device's movements when operated manually by a human operator or via a controller.

The system may also include a robotic device configured to perform tasks autonomously using machine learning policies. The robotic device includes a processor, senor array, and a plurality of actuators, such as servo motors, to enable movement of the device in response to instructions received from the controller. The controller may therefore be configured to process instructions from a machine learning model and based on the processed instructions, drive actuators in the robotic device's joints.

The robotic device may further include a sensor array. The sensor array may include a plurality of sensors configured to capture operational data of the robotic device. The data captured may include joint and gripper positions, external image data of the robot/gripper, and input, such as human corrections applied to the teleoperation device.

As briefly discussed above, in a preferred embodiment, the robotic device may be linked to the teleoperation device such that input on the one may impact the output on the other. In other words, any input from the operator on the teleoperation device may lead to the same movement being executed on the robotic device, or vice versa.

To this end, the system includes a synchronization component that is configured to ensure that the robotic device and the teleoperation device remain aligned. The synchronization component may be configured to enable a plurality of modes of operation. These modes include a robot-driven mode and a human-driven mode. In the robot-driven mode, the machine learning policy being executed drives the robotic device autonomously. In this event, the teleoperation device is configured to mirror the movements of the robotic device. In the human-driven mode, the operator may be driving the teleoperation device, for example by providing an operator input, and the robotic device mimics the resulting trajectories in real-time.

All the components of the system may be in communication with each other directly, or via a server. The server may be configured to maintain or access one or more databases. In particular, the server may have access to (and, preferably maintain), a skill database. The skill database may store one or more refined machine learning policies as reusable “skills”. These skills can be retrieved, deployed, and further augmented through subsequent iterations or interactive data collection, as discussed in more detail below.

The system discussed above may be used to introduce a multi-phase method for interactive data collection and iterative policy refinement. The method may include various stages including, but not limited to, policy execution and synchronisation, human-in-the-loop corrections/adjustments, autonomous operation, data recording and augmentation, policy refinement, and storage of refined policies.

During policy execution and synchronisation, a machine learning policy to be deployed may be selected. The policy may be associated with a task or action to be performed by the robotic device. The policy may be configured to control the morphology-matching teleoperation device. As discussed above, the teleoperation device may be synced with the robotic device and therefore, execution of the policy on the teleoperation device also drives the robotic device. In some embodiments, the policy may be configured to directly execute on the robotic device to control the robotic device, with the teleoperation device mirroring the motion/movement of the robotic device.

The synchronisation between the two devices ensures that the devices always remain aligned, and facilitates human intervention, when required. In other words, any human intervention via the teleoperation device may also result in changes in the movement/motion of the robotic device.

As discussed above, during policy execution, a human operator may intervene to apply corrective adjustments to correct behaviour resultant of the policy being executed.

In a practical implementation, the operator may, for example, switch/actuate/toggle the teleoperation device from an autonomous operating mode to a teleoperation operating mode in which the operator may drive the movements of the robotic device (directly or via the teleoperation device, for example). This may be done via a mode-switching mechanism. The mode-switching mechanism may be a set of physical or virtual switches configured to facilitate the operator to toggle between the two modes of operation. For example, the switch mechanism may be a handheld controller, foot pedal, or button interface mounted on the robotic device. In some embodiments the switch mechanism may be voice operated or configured to interpret gestures, or the like. In even further embodiments, the mechanism may be a software-based switch provided by a user platform.

The teleoperation device may be provided with bidirectional actuators that are configured to enable both passive and active control. This enables the operator to seamlessly take over the device or resume autonomous operation after the operator made an adjustment, for example. The teleoperation device and the robotic device may each include a sensor array. Each sensor array may include a plurality of sensors configured to monitor or collect data relating to one or more of: joins positions, operator inputs and/or robot gripper state and image data. The sensor arrays may be configured to capture the data during any of the two modes discussed above.

Each of the devices may also include a controller in data communication with the respective sensor arrays. The controller may be configured to manage mode transitions, at least process some of the captured sensor data, and synchronise movements between the teleoperation device and the robotic device during operation.

In the autonomous mode, the robotic device (and/or teleoperation device) operates under the control of a trained machine learning policy maintained and stored at a server. The robotic device continuously performs tasks, such as assembling parts, manipulating objects, or navigating an environment, associated with the policy.

The relevant sensor array is configured to record task execution data and logs any deviations from expected performance, such as encountering an edge case or obstacle. The deviations from the expected performance may be flagged manually or, in preferred cases, automatically whenever operator input is required. For example, the sensors and/or controller may be configured to determine when operator input was applied, what type of operator input was applied and send that data to the server for further processing.

When the operator detects suboptimal behaviour or a need for adjustment, they can activate the mode-switching mechanism and intervene. For example, the operator may press a button, tap a foot pedal, or issue a voice command to toggle into teleoperation mode via the teleoperation device.

The transition may be immediate, thereby ensuring no interruption in the task's progress.

Once the mode-switching mechanism is activated, autonomous control is paused, and the teleoperation device switches to a passive mode to allow human-driven control.

In the passive mode, the operator may perform manual corrections by driving the teleoperation device's joints, augmenting the robotic device's behaviour in real-time.

In active mode, operator-applied forces are interpreted as velocity or torque inputs. The corrections are mirrored on the robotic device, enabling the system to adapt dynamically to human guidance.

The system, via the sensor arrays and controllers, records the resulting augmented trajectories of the robotic device, including human corrections. The sensor array may, for example, capture joint positions, image/state data, and any corrective actions. The controller may receive and processes the recorded data for transmission to the server. The processed data is then further processed by the server and aggregated into the existing training dataset for further analysis.

The system and method discussed above therefore supports on-policy learning, ensuring alignment between training and test distributions. The corrective adjustments captured during policy execution are integrated into a training dataset, thereby refining the model under real-world conditions.

The updated training dataset is used to retrain the machine learning policy iteratively. This feedback loop continues until the policy achieves the desired level of performance.

Once the policy reaches the desired level of performance, the policy is stored as a reusable skill in the skill database by the server. These stored policies can be retrieved for deployment or further augmentation by subsequent users, enabling a scalable and collaborative framework for robot learning.

Throughout the specification it should be appreciated that the terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art. For example, unless the context indicates otherwise, the term “policy” and “model” (as it refers to machine learning) should be interpreted to be the same. Even though machine learning policies and machine learning models are related, persons skilled in the art would appreciate that there are differences between these concepts. However, for the purpose of the specification, these terms are used interchangeably.

Given the variety of methods for training AI policies and models and the diverse ways to collect and process data for such training, this disclosure does not attempt to exhaustively detail all possible data collection techniques or training algorithms. Instead, it focuses on describing the overall system's functionality, architecture, and interactions. A person skilled in the art will understand the terminology and appreciate the design and configuration of system components, such as the server, the training processes, and the data-handling mechanisms.

Example embodiments of a system and method for iterative data collection, dataset augmentation, and iterative refinement of machine learning policies for robotic devices will now be discussed with reference to the accompanying drawings.

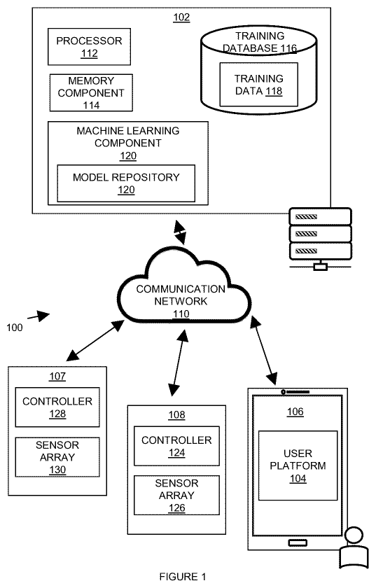

FIG. 1 is a schematic diagram showing an example system (100) for facilitating iterative data collection and refining machine learning policies for robotic devices. The system (100) may include a server (102), a user platform (104), executing on a user device (106), a morphology-matching teleoperation device (107), and a robotic device (108) in digital communication with each other. The server (102) may be under the control and maintained by the same entity maintaining and providing the user platform (104). In some embodiments, the user platform (104) may be integrated into the morphology-matching teleoperation device (107) or the robotic device (108). In such an embodiment a user device (106) may not be present, or the user device may mainly be used as a control or monitoring device.

In a preferred embodiment, the user device (106) is under the same control as the entity (being an operator) controlling the teleoperation device (107).

It should be noted that even though only one user device (106), one teleoperation device (107), and one robotic device (108) are shown, a plurality of user devices, teleoperation devices, and robotic devices may be present in a practical implementation.

The server (102) may be any suitable device configured to interface with the one or more external data sources, not shown. For example, the server (102) may be configured to access robotics training data from one or more external data repositories which may be used to train a robotic device. As will be discussed in more detail below, this data may be used to create and train robot learning models/policies.

The server (102) may be in communication with the user platform (104) via a suitable communication network (110), such as the Internet or any other appropriate communication network, via which data and/or messages may be transmitted to and received. The server (102) may, for example, be configured to receive data packages from the user platform (104), teleoperation device (107), or robotic device (108) that may be used for policy training and generation. Similarly, the server (102) may, in some embodiments, transmit data (including models, for example) and/or notifications to the user platform (104), teleoperation device (107), or robotic device (108). The server (102), teleoperation device (107), robotic device (108), and the user device (106) may include processors capable of processing data, memory units capable of storing data and communication components capable of sending data to other devices and locations.

The server (102) may, for example, be any device capable of performing the role of a server and may be in the form of a cluster of server computers, a distributed server computer, cloud-based server computer, or the like. The physical location of the server may be unknown and irrelevant to users (such as operators) of the system described herein.

The user device (106) may be any suitable computing device such as a mobile phone, tablet, proprietary hardware device, or the like, via which the user platform (104) may function.

As mentioned above, the server (102) may include a processor (112) for executing the functions of its components, which may be provided by hardware or by software units executing on the server (102). The software units may be stored in a memory component (114) and instructions may be provided to the processor (112) to carry out the functionality of the described components. In some cases, for example in a cloud computing implementation, software units arranged to manage and/or process data on behalf of the server may be provided remotely or in a distributed fashion.

The server (102) may have access to (and/or may maintain) one or more training database(s) (116) in which at least robot learning training data (118) may be stored and from which the data may be accessed. The training data may include data acquired from one or more sources, as discussed above. For example, the data may be acquired from public sources, internal databases, or third parties (such as third parties with which an agreement for sharing and acquisition of data is established). Importantly, however, at least some of the training data will be obtained via the user platform (104), the teleoperation device (107), and/or the robotic device (108). The acquired data may include data such as historical operational data, established training datasets, system logs, sensor data, etc.

The data (118) may be used to train or generate one or more robot controlling policies that may eventually be used for supervised or unsupervised control the robotic device (108). For example, the type of data being collected facilitates training and generation processes by allowing the system to employ different training techniques or training paradigms on the data. The data (118) may be used to train or generate one or more robot control policies that may eventually be employed for supervised or unsupervised control of the robotic device (108). For example, as briefly mentioned above, the type of data being collected may include data such as historic robot performance data, newly captured sensory inputs (e.g., images, depth maps, or LiDAR data), or demonstration data from human operators via the teleoperation device (107), or manually. The type of data being collected may be used and processed by the server (102) that is configured to utilise various training techniques or learning paradigms. These paradigms include supervised learning (e.g., training a model to classify objects or predict actions based on labelled data), reinforcement learning (e.g., optimising actions through trial and error using reward signals), or imitation learning (e.g., mimicking expert demonstrations). By leveraging these large, diverse datasets, the server may be configured to adapt its training approach to the specific requirements of a pre-identified robotic action or task. The terms “task” or “action” as discussed herein should be interpreted to be any series of instructions associated with real-life actions, such as detection and movement of the robotic device (108) to achieve a pre-determined objective.

For example, each task or action may be associated with a set of step-by-step instructions used as input to achieve a desired about. A user may, for example, request the robotic device to perform an apple harvesting task. The set of instructions may therefore be configured to enable the robotic device to detect apples on a tree, move the robotic device, or at least its arms closer to the apples, and once in position, actuating grippers at the end of the robotic arms to grip the apple stem and remove the apple from the tree.

Even though the exact robot learning training techniques employed to train the initial models to be used are an integral part of the invention claimed, the Inventor submits that the techniques are well-known in the field of robotics and will not be discussed in detail for the purposes of the description. It should be appreciated that data may regularly be received and processed by the server and, as such, the data (118) may also frequently be updated to include any new, related data.

Given the above, the server (102) may include or have access to a machine learning component (120) which includes a model/policy repository (122) in and from which one or more models may be stored and accessed for use by operators of the teleoperation device and the robotic device. In some embodiments, as discussed in more detail below, well established and confirmed models/policies may be stored in a skills library for access and deployment by users of the system. Users of the system may, for example, be provided access to at least some of the policies in the skills library, via the user platform (104), and which may be used to request the robotic device (108) and/or teleoperation device (107) to perform a task/action associated with said policy.

In some embodiments, although it is not shown, the server may maintain or have access to a user database. The user database may be used to maintain one or more user records. Each of the user records may be associated with a user via a unique user identifier and contain information about the user. For example, each user record may be associated with one or more models/policies in the model/policy repository so as to indicate whether the user may be provided access to, or request an action associate with, a particular policy. A subscription model may, for example, be provided which requires users of the system to subscribe to a service in order to obtain access to models and/or policies. The policies stored in the skills library may, for example, be purchased by users of the system.

The user device (106) may include a processor for executing the functions of the user platform (104) which may be provided by hardware and/or by software units executing on the device (106). The software units may be stored in a memory component and instructions may be provided to the processor to carry out the functionality of the components of the platform in cooperation with an operating system of the device. In some cases, for example in a cloud computing implementation, software units arranged to manage and/or process data on behalf of the mobile electronic device may be provided remotely. Some or all of the components may be provided by the user platform (104) that may be downloadable onto and executable on the respective user device. In some embodiments, the user platform (104) may be a web-based application accessible by the user device without requiring the user to have the platform installed on the user device. In other words, the user platform does not need to be limited to a downloadable software application that is resident on the user device. And, it may, be a web-based application provided and maintained by the server.

The user platform (104) may be configured to enable a user to record data for training and generating robot learning policies to control the teleoperation device (107) and/or the robotic device (108). The platform may, for example, include a user interface and be configured to interact with other software and/or hardware components of the user device (106).

In some embodiments, the user platform (104) may be used to control the robotic device (108) directly or send instructions to the robotic device. It should be appreciated that any data exchanged between the robotic device and the user device can occur either directly between the devices or, in some embodiments, via the server. In the latter case, the server may at least temporarily store the data during the exchange. In a practical implementation, for example, a user of the operating device may desire an action to be performed by the robotic device. The user may request the action to be performed via the interface of the user platform (104). In a preferred embodiment, this request is transmitted to the server and processed. Once processed, the server may transmit an activation signal or instruction to the robotic device (108) to perform the desired action. In some embodiments, however, the request may directly be transmitted to the robotic device (108), where the request is also processed. Once processed, the robotic device may query the server to determine of the user has access to the requested action, and, if so, the robotic device may access one or more locally stored models for deployment. Alternatively, the robotic device may request access to one or more models stored at the server (102) for deployment.

In some embodiments, the user platform (104) may be used to control, or at least activate, the teleoperation device (107) to control the robotic device (108). For example, the user may request the action to be performed via the interface of the user platform (104). The request is transmitted to the server and processed. Once processed, the server may transmit an activation signal or instruction to the teleoperation device (107). The teleoperation device may be activated and used to control movement of the robotic device (108), as discussed in more detail below.

Importantly, the user platform (104) may be configured to allow a user/operator of the teleoperation device (107) or robotic device (108) to capture image data (in the form of videos and/or photos) via a camera of the user device (106). The image data may be used to capture details of the environment or an operator that may be used to train and/or generate policies. For example, the images may be used for supervised learning by extracting features to identify objects, recognise patterns, for classify scenes. In some embodiments, the image data may be used to capture certain states or observations that can be mapped to corresponding actions, facilitating imitation learning. In such cases, the policy is trained to replicate the behaviour demonstrated in the dataset to, for example, allow the robotic device to learn tasks efficiently by observing human or expert behaviour.

In some embodiments the user platform (104) may be configured to, via a processor of the user device, at least pre-process the captured image data. The user platform (104) may, for example, be used to capture the image data through a camera or other sensors of the user device (106) in real-time or as part of pre-recorded intervals. The captured data may then be processed through a pre-trained model or algorithm (using computer vision techniques, for example) to extract relevant features. This may include object detection, classification, segmentation or other image analysis tasks. In some embodiments, the user platform may call the models used to process the data from the server, periodically or on demand.

In some embodiments, the processed data may then be sent to the server for use in further training of the robot learning models/policies.

In a preferred embodiment, any complex processing techniques, such as computer vision, for example, will be performed by the server instead of the user device (106).

In some embodiments, the user platform (104) may send instructions to one or both of the teleoperation device (107) and the robotic device (108) to perform an action based on the output associated with the processed data.

This parsed data may then be used to create a data package that may be sent to the server (102), teleoperation device (107) and/or the robotic device (108).

The robotic device (108) may include a plurality of electro-mechanical components, such as servo motors, gripping actuators, or the like. In addition to these components, the robotic device (108) may include a controller (124) configured to control the components of the robotic device (108). For example, the controller may be configured to receive instructions from the server (102), user platform (104), or teleoperation device (107), process the instructions, and transfer one or more action signals to the components of the robotic device (108). The robotic device (108) may further include a sensor array (126). The sensor array (126) may be configured to record/monitor data associated with the environment, with the robotic device (108) relative to the environment, or with the movement of the teleoperation device (107), during operation. In particular, the sensor array (126) may be configured to record a stream of visual information and joint information associated with arms and grippers of the robotic device (108). The sensor array (126) may therefore comprise of one or more visual sensors and joint position sensors. The robotic device (108) may also include local storage where models/policies associated with robotic actions can be stored, at least temporarily. For example, if a particular robotic device is always used for the same task/action, the device may be configured to at least temporarily store the model associated with the particular action to avoid unnecessary communication (which may cause delays) between the server and the robotic device.

The controller (124) of the robotic device (108) may further be configured to control components of the robotic device, as mentioned above. In other words, the robotic device (108) may also operate independently and autonomously if desired. In such scenarios, the robotic device (108) may be driven by the relevant trained robotic policy. It should be appreciated that the robotic arms may be placed on a support structure, such as a stand or tabletop, allowing for autonomous operation without any direct involvement from an operator.

The controller (124) may further be in communication with one or more of the server (102), the user platform (104), and/or the teleoperation device (107). This communication enables instructions and data to be exchanged between these components of the system (100). For example, the controller may be configured to facilitate control of components of the robotic device during autonomous operation thereof. In some embodiments, the server may be configured to collect/receive and process data from the sensor array (126) of the robotic device (108), and transmit the data to a controller of the teleoperation device (107) for processing.

The teleoperation device (107) may be a robotic type of device configured to be operated by an operator. Preferably the teleoperation device may be operated, or at least partially operated, by direct human-controlled movements.

The teleoperation device (107) may also include a controller (128), similar to the controller (124) of the robotic device (108), configured to control/engage components of the teleoperation device (107). In a preferred embodiment the teleoperation device is a duplicate version of the robotic device (108) with minor adjustments. For example, the teleoperation device (107) may have different dimensions and be configured to be secured to an operator for operating the device (107).

The teleoperation device (107) may further include a teleoperation sensor array (130) configured to record/monitor data. Preferably, the sensor array is configured to record data associated with movement of the teleoperation device. As discussed above, in a preferred embodiment, the teleoperation device is a teleoperation mechanism configured to closely replicate the kinematic chain of a target robotic system, which in this case is a robotic device (108). The teleoperation device (107) may include one or more portions including joints (for example articulated robotic arms) and each joint of the teleoperation device may include a sensor, such as a rotary encoder, arranged to measure joint positions and actuators arranged to enable motor-driven movements of the joints of the teleoperation device (107).

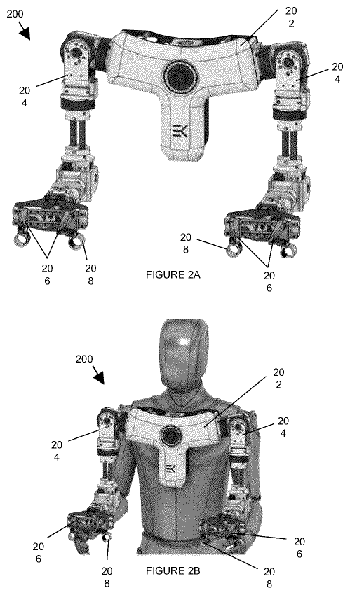

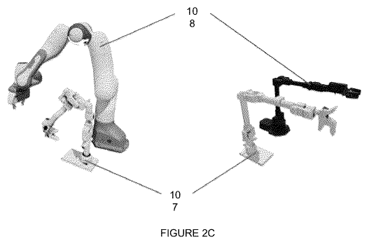

FIGS. 2A and 2B show an example embodiment of a unified robot and teleoperation device. FIG. 2A shows the device in isolation, where it can operate as a robot, whereas FIG. 2B shows the device worn by a user/operator of thereof where it acts as a teleoperation device that the user can control to collect data. In the examples shown, the device has a substantially exoskeleton-like configuration enabling the device to be worn by an operator as is clear from FIG. 2B.

The exoskeleton (200) may be secured to an operator via any suitable means (not shown). For example, in a practical implementation, the exoskeleton (200) may be removably secured to the operator via one or more straps configured to engage with the user.

The exoskeleton (200) includes a central body (202) configured to house some of the components of the device such as the controller (128).

The exoskeleton (200) may include two arm portions (204) with each arm having a gripping member (206) at a free end thereof. Each of the gripping members (206) may include an override mechanism (208) configured to receive fingers of an operator (for example an index finger and thumb) that may be used to manually control/steer the gripping members. This may enable the operator to manually apply adjustments or perform corrective actions that may be recorded by the sensors of the exoskeleton.

When acting as a teleoperation device, as shown in FIG. 2B, the device may be configured to enable direct human/operator intervention and manipulation of movement of the teleoperation device, as discussed in more detail below. When the teleoperation device is integrated with an operator, the robotic arms may be attached to a torso structure (being the central body), secured with adjustable straps to ensure precise mirroring of the operator's movements. The sensor system continuously records visual and positional data, which may later be used to train a robot learning policy. The corrective adjustments or corrective actions performed by the operator may therefore include the operator, for example, manually moving the arms, of the device in order to demonstrate a desired movement. The corrective adjustments may therefore be any physical input manually provided by the operator to manipulate the movement of the device.

In some embodiments, the corrective adjustments may be remotely provided by the operator via the user platform (104). For example, the operator may request movement of the robotic arm, in response to visually detecting that the teleoperation device is not moving as desired. The operator input may therefore alter an undesired output of the teleoperation device by overriding the automatic/model-controlled output and demonstrating a desired output by enabling an operator to manually adjust and control the teleoperation device's movements via the user platform (104) or physically. These adjustments are recorded and may be sent to the server where it can be processed and used to further refine the robot's learning policy, enhancing its performance in unpredictable environments.

As discussed above, the teleoperation device (107) and robotic device (108) may have similar configurations and be linked to one another. The link between the devices may be configured to enable movement of one device to impact or influence movement of the other device. For example, the teleoperation device (107) may be controlled by an operator to perform a selected action. The teleoperation device (107) may be configured to perform the action autonomously, semi-autonomously, or completely human controlled. During performance of the action, the sensor array (130) of the device (107) may capture all movements of the teleoperation device (107). Those movements may be recorded, processed and sent to the controller (124) of the robotic device (108), either via the server (102) or directly from one controller (128) to the other (124). The robotic device may therefore be configured to mirror the movements of the teleoperation device (108). In such an embodiment the robotic device's trajectory and movements may be captured/recorded using the relevant sensor array and sent to the server where it may be sed to further refine the robot's learning policy.

In another embodiment, the vice versa may also be true. In other words, the system may be configured to enable the robotic device (108) to perform a predetermined task. The robotic device (108) may autonomously perform the task, via instructions sent from the server to the controller, for example. The sensor array (126) may be used to capture data associated with the movement of the robotic device (108). This data may be recorded, processed and sent to the controller (128) of the teleoperation device (107) either directly or via the server (102). The teleoperation device (107) may receive and process the data and perform the same movements as the robotic device (108). In such an embodiment, it is important that the teleoperation device (107), or at least the actuators thereof, is back-driveable to enable the operator to counter some of the movements mirrored by the teleoperation device (107). These corrective movements may be recorded and processed to be used for training, as discussed in more detail below.

In light of the above, the teleoperation device (107) may further be configured to support both passive and active control modes. In the passive mode, the operator of the device (107) may directly drive the joints of the device (107) while the actuators thereof move freely. In other words, as mentioned above, the actuators may be configured to enable free, low friction movement of the joints when the teleoperation device (107) is in the passive mode. In the active mode, one or more torque sensors of the sensor array (130) provided on the teleoperation device (107) may be configured to detect operator input forces, and the controller (128) (or, in some embodiments, internal controllers of the actuators) applies velocity or torque commands to the actuators. In other words, in the active mode, the user and robotic device essentially both moves components of the device at the same time.

It should be appreciated that, in some embodiments, components of the user device (such as cameras, accelerometers, or the like) may be provided by the teleoperation device or the robotic device itself. In such embodiments, the user device (and user platform) may be configured to simply transmit remote instructions to control the teleoperation device and/or robotic device. In other words, in some embodiments the user platform (104) is simply a control and monitoring platform. In these embodiments, the data captured using the user platform, discussed above, may be transmitted to the teleoperation device, robotic device, and/or the server where it may be processed.

It should be appreciated that in some embodiments the robotic device and teleoperation device may be a single device. In such cases there no need for synchronous movement between the two different devices and the same device can operate as both a robotic device when left to stand alone (for example using a similar device to the device shown in FIG. 2A) or a morphology matching teleoperation device when worn by the user and exoskeleton as shown in FIG. 2B.

In another embodiment the two devices are separate entities as shown in FIG. 2C. The morphology matching teleoperation device (107) in this case can communicate with the robotic device (108) via the server (102) allowing the two devices to operate synchronously by sharing joint positional information.

It is an object of the system described herein to enable a user/operator thereof to assist in the training, adjustment, and refinement of robot learning policies and also providing a user/operator access to established policies and to access said policies from the server and, via the user platform, instruct the robotic device to operate in accordance with the policy. In other words, the more operators make use of the system, the more policies may be created and refined for use in specific tasks to be performed by the robotic devices. The server may be configured to store these refined policies (based on a score, for example) and make these policies available to new or existing users based on pre-established conditions. Different users may therefore request policies from the server and, if provided access, download or access the policies via the user platform and instruct the controller of the robotic device to act in accordance with the policies. The users may adjust the policies as desired.

In an example embodiment, the system may perform methods steps in different phases, including a data collection phase, autonomous operation and learning phase, and a human intervention and adjustment phase.

Each of the phases and what they include are briefly set out below:

### Data Collection Phase

In this phase, the sensor array (126, 130) of the robotic device (108) and the teleoperation device (107) are configured to record/collect a continuous stream of visual data (e.g., RGB-D, LiDAR) and the joint configurations of the robotic arms of the relevant devices. The operator, can freely move the teleoperation device to in turn control the robotic device (108) to complete tasks, with the robotic arm motors (actuators) configured to match the teleoperation device's (107) configuration and/or movement. This collected data may be stored, either in an on-board memory of the controller (128), or at the server (102), for later preprocessing and use in training machine learning models. In the case where the teleoperation device and the robotic device are unified, as in FIGS. 2A and 2B, the human/operator can directly backdrive the device to complete a task and the respective state data including images and joint positional data are stored.

### Autonomous Operation and Learning Phase

The collected data may be utilised to train machine learning models to obtain a robot learning policy, using techniques such as imitation learning or reinforcement learning (as discussed above). Once the policies have been sufficiently trained/obtained, the system may operate autonomously, either with or without direct operator interaction, depending on the task requirements.

### Human Intervention and Adjustment Phase

During autonomous operation, the system is configured to enable the operator to intervene and correct the robotic device's (108) behaviour in real-time, via the teleoperation device (107). The adjusted behaviours can be recorded via one or both of the sensor arrays (126, 130) and added to the data store to refine the robot's learning policy further.

During the initial data collection phase, the server (102) may collect data from demonstrations conducted by experts, real-world robot trials, for example using the teleoperation device (107) and the robotic device (108), simulations, and/or other available data stores.

This collected data may then be processed by the server (102) to generate one or more training datasets (118), which may include labelling the data, formatting the data and cleaning the data, or the like. The training datasets (118) may be stored in the training database (116), as discussed above.

Once the data has been collected and pre-processed, the server may be configured to use the training data (118) to train one or more initial machine learning models using suitable learning techniques. Each of the one or more models may be associated with a particular task or action. For example, the server (102) may be configured to use some of the demonstrations conducted by experts for imitation learning. Once a model has been trained, the model may be packaged and stored in a model/policy repository (122) for deployment.

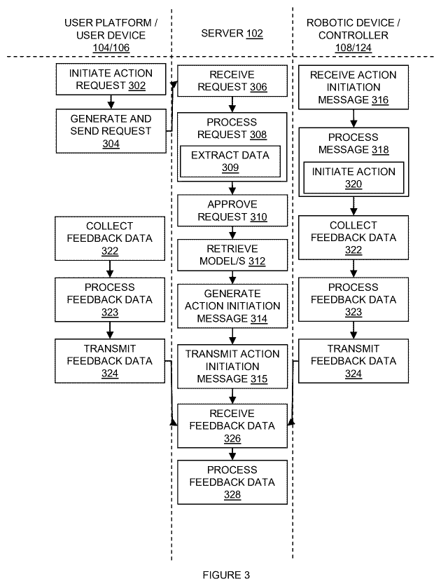

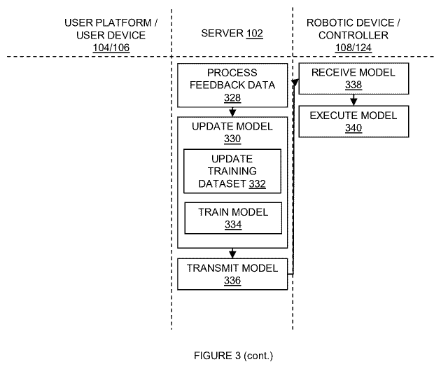

FIG. 3 is a swim lane-flow diagram showing an example embodiment of a method for interaction data collection, dataset augmentation and iterative refinement of a machine learning policy. It should be appreciated that the method shown is an example method only and various embodiments may be envisaged that falls within the scope of the claimed invention.

In other words, the system (100) may have different modes of operation based on the components within the system. For example, the system may preferably operate in one of the following scenarios: i) operator-driven policy augmentation; ii) robot-driven operator corrections; or, iii) a unified robot-teleoperation device for corrections.

In the operator-driven policy augmentation embodiment a machine learning policy may be deployed to operate on the teleoperation device (107). The robotic device (108) may be configured to track and mirror the teleoperation device's movements. The human operator may intervene to adjust the teleoperation device (107) so as to correct deviations in the behaviour of the device (107) compared to the expected behaviour. The corrected trajectories or movements are recorded via the relevant sensor array (126, 130), processed, and aggregated into the training dataset. This data may then be used to retrain the policy.

In the robot-driven operator corrections embodiment, the machine learning policy may be deployed and executed on the robotic device (108), autonomously. The teleoperation device (107) may be configured to track and mirror the robotic device's (108) movements. The human operator may, when inaccuracies or undesired operation is detected, intervene and apply manual corrections via the teleoperation device (107). These augmented trajectories are recorded via the relevant sensor array (126, 130), processed, and used for on-policy refinement.

In the embodiment where a unified robot-teleoperation device is used for corrections, the teleoperation device (107) and the robotic device (108) may be a single device. The system may be configured to alternate seamlessly between autonomous operation and teleoperation (where human input is received). Human corrections during autonomous execution may be recorded via the sensor array, processed and eventually used to train the machine learning policy. This configuration of the system may simplify the hardware requirements of the system and enhance usability by enabling direct human interaction with the robotic system.

The swim-lane flow diagram of FIG. 3 shows an example method of a system in which a unified robot-teleoperation device is used. In other words, the embodiment to which the method relates is an example embodiment in which the teleoperation device (107) and the robotic device (108) are integrated into a single device. It should be appreciated that, as clearly discussed above, this is just one example embodiment of the system. The purpose of FIG. 3 is to indicate an example flow of the data between the devices/components within the system. For the sake of simplicity, the robotic device (108) with reference to FIG. 3 should be interpreted to mean the unified device provided by the combination of the robotic device and the teleoperation device.

The swim lane-flow diagram shows steps in which the user platform (104) mainly communicates with the robotic device (108) via the server (102). In some embodiments, however, it should be appreciated that the user platform (104) may communicate directly with the robotic device (108).

It should also be appreciated that the server (102), in the present example, already has access to one or more initially trained machine learning models stored in a model repository (122). In a practical implementation training one or more machine learning models may be the first step in the system. The models may be trained using a series of collected data, mentioned above. It should be appreciated that the initial models may be trained using known, conventional training methods.

A user, which may also be the operator discussed below, may initiate/request (302) an action to be performed by the robotic device (108) via the user platform (104). The request may be submitted in order to train a specific policy, or to use the robotic device for a specific ask. Initiating/requesting (302) the action may include the user selecting or inputting an action to be performed by the robotic device (108) into the user platform (104) via the user interface. The action may, for example, be selected from a list of actions. The list of actions may be provided and maintained by the server (102) and associated with a particular machine learning model/policy. In some embodiments, the list of actions may be unique to the user and associated with a user record.

Once the user has selected an action via the user platform (104) a request may be generated and sent (304) to the server (102). The request may be in the form of a data message and at least include an identifier associated with the user of the user platform (104) and an identifier/data associated with the selected action.

The server (102) may receive (306) and process (308) the request. Processing (308) the request may include the server (102) extracting (309) the information/data from the request to identify the model associated with the requested action and whether the user associated with the user platform (104) has access to the requested action.

In some embodiments, as suggested above, the server (102) may maintain one or more user records in the database (118). A user record may be created for users that are registered to use the platform (104). In some embodiments users may be required to register via a registration process to use the platform. This registration process may be any standard registration process and will not be discussed herein in detail.

Each user record may be associated with one or more action identifiers. And each of the one or more action identifiers may be associated with a particular machine learning model/policy and action. In a practical implementation, a user may, for example, register to gain access to a particular action in exchange for a monthly or once-off payment.

In other words, the server (102) may have access to one or more user records, where each user record is associated with a user identifier and one or more action identifiers. Each of the one or more action identifiers may be associated with one or more machine learning models/policies maintained in the model/policy repository (122).

Turning back to the method, processing (308) the request and extracting (309) the information from the request may include the server (102) identifying the model/policy associated with the action identifier and determining whether the user, associated with the user identifier, has access to a model/policy associated with the action identifier. This may include the server (102) comparing the user identifier with an expected user identifier, and if the user is the expected user, determining if the action identifier included in the request is an action identifier associated with the user record.

If it is determined that the user does not have access to the requested model associated with the action, the server (102) may return a rejection message or notification to the user platform (104). In a practical implementation this rejection message or notification may include an option to purchase the action or access the action by providing some additional information, for example.

If it is determined that the user does have access to the model associated with the action identifier, the server (102) may approve (310) the request and retrieve (312) the one or more models from the model repository (122). Once the models have been retrieved, the server (102) may generate (314) an action initiation message and transmit (315) the action initiation message to the controller (124) of the robotic device (108). The action initiation message may include the retrieved model/s (or at least a pointer to the model/s) associated with the action identifier.

The controller (124) may receive (316) the action initiation message and process (318) the message. Processing the action initiation message may include initiating (320) the requested action and applying/executing the one or more models to process inputs and control components of the robotic device (108), such as actuators.

Once the action is initiated, the sensor array (126) of the robotic device (108) may collect (322) behavioural/feedback data of the robotic device (108). The data collected by the sensor array (126) may be returned to and processed (323) by the controller (124).

This may include the controller converting the data to a desired format, interpreting the data, and then sending instructions to the components of the robotic device (108). For example, if the sensor array detects minor issues, such as the robot arms failing to locate an object to grip, the controller may be configured to apply local adjustments, such as re-calibrating the sensors or retrying the failed action.

The sensor array may be configured to continuously capture data during execution of the action and focus on conditions such as edge cases, failure scenarios, variations in the environment, human interaction, or the like.

Processing the data collected via the sensor array (126) may include the controller creating data logs and metrics for processing by the server (102). The controller (126) may transmit (324) the processed feedback data collected from the sensor array (126) to the server (102). The server (102) may receive (326) and process (328) the feedback data.

Processing (328) the feedback data may include converting the data into a readable format and analysing the data to identify any corrective adjustments performed by an operator and, in some embodiments, corrective adjustments and any additional operating data, such as robot state and image data. The data processed by the server may then be used to update (330) the model associated with the requested action. Once the model has been updated, the server (102) may again transmit (or in another way make available) the updated model to the controller (124) for execution. Processing the feedback data by the server may include aggregating the training dataset associated with the machine learning model with the analysed feedback data.

In other words, as the robotic device (108) executes the steps associated with the requested action, the sensor array (126) collects new observation data and logs situations where the current model/policy leads to suboptimal actions/execution. This collected data is sent to the server where it may be reviewed by a user. The user may provide corrections for these edge cases and add them to the dataset. In some embodiments, where an operator is wearing the robotic device (108), the operator may provide real-time adjustments/corrections when the robot deviates from the desired behaviour. These corrective inputs may be recorded via the sensor array, alongside the other observations, processed and added to the dataset, thereby enriching the dataset.

The server (102) may retrain/update (330) the model using this expanded, aggregated dataset to incorporate both previous experience and the newly collected corrective feedback. Updating the model may therefore include updating (332) the training dataset of the model with the expanded, aggregated dataset (including the feedback data) and using this data to train (334) the model.

Once updated, the model may be redeployed (336) by the server (102) to the robotic device (108) for execution. The robotic device (108) may receive (338) the model and execute (340) the model. The feedback loop may then be repeated as needed.

In some embodiments, the user platform (104), and by extension the user device (106), may form part of the sensor array (126) and be configured to capture operating data when the robotic device (108) is in use. The mobile device's (106) camera and other internal components may be used to capture data and transmit the data directly to the server, or to the server via the controller. In other words, the controller (124) may be configured to collect data from all the sensors in the sensor array (including the user device (106)), process the data, and then transmit the data to the server.

In some embodiments, the controller may be configured to send the captured data to the server in response to a corrective action being detected. For example, as discussed above, the robotic device may be back-driven enabling a user to demonstrate and/or correct the robot's behaviour. This data is monitored and captured by the sensor array and controller and may be used to update the models. The controller may be configured to start a detailed data recording sequence once any back-driven input from and operator is received, and once the input stops, the data may be processed and sent to the server, for example. In some embodiments, data may continuously be collected, logged and sent to the server at pre-determined intervals.

Accordingly, based on the above, the system includes a training phase, during which a user may capture and/or obtain training data used to train one or more machine learning models with each model being associated with one or more actions.

Once the models have been subjected to some initial training, the robotic device may be used in autonomous or semi-autonomous applications. In such embodiments, an operator may wear or otherwise engage with the robotic device, and request the robotic device to perform an action (preferably via the user platform).

This instruction is sent to a server which is configured to process the request and determine if the user associated with the request has access to the requested action. If the user does have access to the requested action, the server transmits the model associated with the action to the controller of the robotic device for execution. In some embodiments the term “transmit” should not be taken literally, and the server may only make the model available for use by the controller of the robotic device.

Once the action is executed or initiated by the controller, the controller may also start collecting data from a sensor array of the robotic device and/or user device. The sensor array may include one or more optical sensors, rotary encoders, or the like. The sensors may be configured to capture state information, such as images and video, and physical information, such as the joint and gripper positions of the robotic device. The robotic device may be configured to be back driveable, so that the operator may alter and override any pre-configured movement of the robotic device brought about by the execution of the model.

In other words, the operator may monitor or capture the behaviour of the robotic device using the sensors of the device itself together with the user platform. The operator (or an additional user) may use the platform or robotic device itself to adjust the behaviour of the robot. This may, for example, include performing remote adjustments, simply recording errors, or showing the correct behaviour.

This collected data may be recorded/logged, packaged, and shared with the server, where the data may be aggregated with previous data sets associated with the current policy/model being trained.

In some embodiments, and as discussed in more detail below, it should be appreciated that in a practical implementation the data collected may constantly be supervised. This supervision may be manual supervision by a human operator, or via algorithms executing on the server.

The supervisor or server may, based on the data collected during operation of the robotic device, determine if the actions performed by the robotic device associated with the executed model/policy has a performance above a certain pre-determined threshold. For example, the server or human supervisor may evaluate the corrective inputs provided by the operator during operation of the robotic device. The sum of inputs may be given a score, and that score may be used to determine a performance rating of the model/policy. If a policy/model is determined to have a performance of above a predetermined threshold, such as 98%, for example, the policy/model may be marked as ready for deployment.

In some embodiments, this may include recording the policy as a skill and storing the policy in a skill database. It should be noted that even if a policy has a sufficient performance score to be considered a skill, the policy/model may still be trained and improved using the above training techniques.

If the model does not meet the pre-determined threshold, the model may be stored separately and required to undergo further training.

As discussed above, the system may also be configured to include a teleoperation device (107) that is a separate device used to train the robotic device (108). The teleoperation device (107) may essentially perform similar steps to the robotic device (108) discussed with reference to FIG. 3. A major difference in such an embodiment, however, is that the teleoperation device (107) may be configured to communicate with the robotic device (108) and the devices may be synchronised with each other.

In use, the teleoperation device (107) may be configured to mirror the movements of the robotic device (108), but enable an operator thereof to interact with the teleoperation device (107) and thereby provide corrective feedback to be used for aggregating the training dataset. In some embodiments, the teleoperation device may be configured to perform the functions of a machine learning model/policy and the robotic device (108) is configured to map and mirror the movements of the teleoperation device. In such an embodiment, the operator of the teleoperation device (107) may still be able to interject and adjust the movement of the device (107) if it is clear to the operator that the device (107) is performing a task or action in an undesired manner. In such an embodiment, mechanical components of the teleoperation device (107) may be back-driveable so that the operator may override the automated movements.

In other words, in an embodiment where the teleoperation device (107) and the robotic device (108) are two separate devices, at least one of the devices may be configured to mirror the movement of the other device. Additionally, the teleoperation device (107) and/or the robotic device (108) should be configured to record any operator input that may be provided to adjust the behaviour of the relevant device.

In an example embodiment, an operator may request a task/action associated with a policy/model to be performed from the server (102), via the user platform (104). The policy may be accessed by the controller (128) of the teleoperation device (107) and the policy may be executed autonomously. The sensor array (130) may be configured to capture any data after initiation of the processes. The robotic device (108) may be configured to mirror the movement of the teleoperation device (107).

If any inaccuracies or mistakes are detected by the operator during execution of the policy, the operator may interject. This may include the operator, at least temporarily, pausing execution of the executing policy, via a predetermined input, and manually adjusting the movement of the teleoperation device (107). The sensor array (130), together with the controller (128), may be used to record and process these movements. The robotic device (108) may also mirror these operator provided inputs and, in some embodiments, the sensor array (126) of the robotic device (108) may be configured to record and process these operator inputs.

The training dataset may then after a predetermined period of time, such as after successful execution of the task associated with the executed policy, transmit the recorded and processed data captured by the relevant sensor array to the server (102) for further processing and aggregation into the training dataset. The robotic device (108) may be configured to track any and all movement of the teleoperation device (107) during execution of the policy. In other words, the robotic device (108) may also adjust its movements with any user/operator input.

The controllers (124, 128) of the respective devices (107, 108) may therefore be in data/network communication with each other to transfer sensor data and/or movement instructions between the devices. In some embodiments, the sensor array of the one device may be configured to track movement of the other device and simply replicate the movement. This may be done via markers, trackers, or the like. In such embodiments the controllers may be configured to receive the sensor data, process the data, and output instructions to components of the relevant device.

In some embodiments, the teleoperation device (107) may operate in different modes, based on the requirements of the system. For example, the teleoperation device (107) may include a mode-switching mechanism configured to enable the operator of the device (107) to switch between autonomous and teleoperation modes. In the autonomous mode, the device may be controlled by a machine learning policy and perform tasks without requiring any human intervention. In the teleoperation mode, the device may directly be controlled by a human operator for task execution or data collection, as discussed above.

The operator may manually toggle the mode-switching mechanism. Once the mode-switching mechanism is activated, autonomous control is paused, and the teleoperation device switches to a passive mode to allow human-driven control.

In the passive mode, the operator may perform manual corrections by driving the teleoperation device's joints, augmenting the robotic device's behaviour in real-time. These operator movements may be recorded and processed and transmitted to one or both of the server (102) and the controller (124) of the robotic device (128). The controller (124) may then instruct the components of the robotic device (108) to mirror the movements of the teleoperation device (107).

In active mode, operator-applied forces are interpreted as velocity or torque inputs. The corrections are mirrored on the robotic device, enabling the system to adapt dynamically to human guidance. Similar steps may therefore be followed in both modes (active and passive mode), but the components of the device may be configured differently.

As discussed above with reference to FIG. 3, these human operator interactions may be recorded and processed by the system and augmented into the training dataset.

FIG. 4 is a flow diagram illustrating an example method for determining a performance score of a model. The steps of the method may be performed whenever the model is executed in response to an action request.

The steps are performed at the server (102).

During initial creation of the dataset and training of the models, the data may also include / outline what constitutes a successful execution of the action associated with a particular model. In other words, a user may define success criteria for the action, and this may be stored at the server and associated with the particular model.

The server (102) may receive (402) a set of feedback data from any one or more of the user platform (104), the teleoperation device (107), and the robotic device (108). The set of feedback data includes pre-processed data logs based on the interactive data captured/collected via the relevant components of the device/s, such as a sensor array (126, 130).

The server (102) may process (404) the feedback data. Processing (404) the feedback data may include the server analysing (406) the feedback data to determine whether the robotic device (108) achieved a desired action objective. This may include, for example, determining if any corrective actions where required during the execution of a model on the teleoperation device (107), or robotic device (108). If any corrective actions where required, the server may identify the type of corrective actions required from the feedback data.

In some embodiments, it should be appreciated that processing (404) and analysing (406) the feedback data may include the server (102) identifying a record for storing of the feedback data and storing the feedback data as a data set/data stream in the record. In other words, the received feedback data may include the entire trajectory performed by the relevant device and the server does not need to distinguish between different types of data or extract specific data from the feedback data.

Each type of corrective action may, in some embodiments, be associated with a particular score indicative of the success or failure of an action. The more complex/impactful the corrective action, the higher the score associated with the action. Similarly, the simpler/less impactful the corrective action, the lower the score associated with the action. For example, a complex corrective action may be to completely adjust the orientation of the robotic device. A simpler corrective action may, for example, be to slightly adjust the gripping strength of the grippers of the robotic device (108). This may be via the teleoperation device (107) or, in the case where the robotic device (108) and the teleoperation device is a single device, the robotic device (108) discussed with reference to FIG. 3.

The data logs may further include data such as completion time, failure modes, or the like.

Once the data has been processed and analysed, the server (102) may determine (408) a model performance score associated with the model. Determining (408) the model performance score may include matching the robotic device's actions in the data logs (or the scores of types of corrective actions) with the predefined success criteria. The performance may be calculated as a ratio of successful actions to the total number of actions executed/performed.

If the model performance score is above a predefined threshold, for example 98% (however it should be appreciated that the required performance score may differ on a case-by-case basis), the server (102) may consider the model/policy (or a copy thereof) as a skill and store (410) the model in a skill database, for example.

If, however, the model performance is below the predefined threshold, the model is not stored in the skill database. The model may then be required to undergo further refinement and testing. Various components may be provided for implementing the method steps described above.

FIG. 5 is a block diagram which illustrates exemplary components of a server (102) which may be provided by a system for facilitating behavioural/interactive data collection to be used to train robot controllers.

The server (102) may include a processor (112, 502) for executing the functions of components described below, which may be provided by hardware or by software units executing on the computing device. The software units may be stored in a memory component (114, 504) and instructions may be provided to the processor (112, 502) to carry out the functionality of the described components. In some cases, for example in a cloud computing implementation, software units arranged to manage and/or process data on behalf of the server (102) may be provided remotely.

The server (102) may include a request receiving component (506) arranged to receive, via a user platform (104) on a user device (106), a request for initiating an action to be performed by a robotic device (108). The request may include data associated with the user (or user device) and an action requested to be performed by the robotic device (108). The request may, for example, include a user identifier and an action identifier.

The server (102) may include a request processing component (508) arranged to process the request so as to extract and identify data from the request that may be used in further processing. The processed data (being the extracted and identified data) may be, at least temporarily, stored in the memory (504) of the computing device, and used to identify a machine learning model associated with the requested action, and to determine of the user has access to the requested machine learning model.

The server (102) may include a machine learning component (510) arranged to provide access to a machine learning model, associated with the requested action, to a controller of one or both of the robotic device and the teleoperation device. In particular, the machine learning component may have access to a model repository where trained (or semi-trained) machined learning models are stored for use. Different models in the model repository may be configured to provide different outputs in response to receiving different inputs. For example, different models may be associated with different actions. The machine learning component (510) may further be arranged to instruct or enable the controller (124, 128) of the relevant device to execute the model.

The server (102) may include a feedback receiving component (512) arranged to receive feedback data from at least the controller of the relevant device. In some embodiments, the feedback receiving component (512) may also be configured to receive feedback data from the user device (106), via the user platform (104). The feedback data may be data recorded/collected by a sensor array (126, 130) of one or both of the devices. The sensor array may include one or more sensors, actuators, provided on the robotic device (108) or the teleoperation device (107). The feedback data may be processed by the controller into data logs that can be interpreted by the server (102). In particular, the feedback data may be indicative of operator input or corrections applied to the movement of the robotic device during operation thereof.

The server (102) may include a feedback processing component (514) arranged to process the feedback data. The feedback processing component (514) may analyse the feedback data to identify any corrective adjustments performed by an operator of the device. This may include parsing the data logs associated with the feedback data to determine if there were any corrective adjustments.

The server (102) may also include a machine learning model updating component (516) arranged to update the machine learning model based on the feedback data. The machine learning model component may use the processed feedback data and aggregate an initial training dataset associated with the model with the feedback data. This new aggregated set of training data may then be used to re-train the machine learning model.

The server (102) may further include a model performance score determining component (518) arranged to determine a performance score of the model being executed. The performance score determining component (518) may be used to compare the actual output of the model, using the feedback data, to a desired or expected output of the model. The result of the comparison may be expressed as a ratio of successfully completed actions against the total number of actions associated with the model. Once the ratio has been determined, a performance score may be determined based on the ratio. If the performance score is above a certain threshold, the machine learning model may be stored in a skills database.

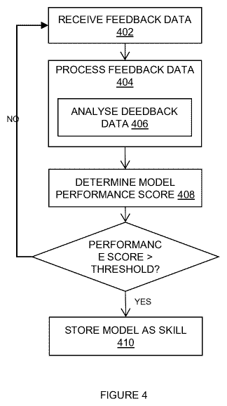

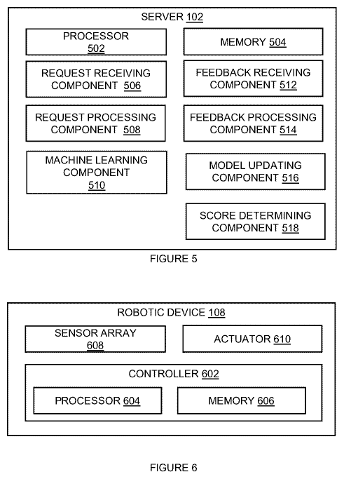

FIG. 6 is a block diagram which illustrates exemplary components of a robotic device (108) which may be provided by a system for facilitating behavioural/interactive data collection to be used to train robot controllers.

The robotic device (108) may include a controller (124, 602) arranged to receive instructions and instruct components of the robotic device (108). The controller (124) may include a processor (604) for executing the functions of components described below, which may be provided by hardware or by software units executing on the controller. The software units may be stored in a memory component (606) and instructions may be provided to the processor (604) to carry out the functionality of the described components.

The robotic device may include a sensor array (126, 608) arranged to record/collect feedback data, such as robot state information including joint and gripper positional data and image data of the task, during operation of the robotic device (108). The sensor array (126, 608) may include one or more sensing devices which are all configured to track movement of the robotic device, image state data, or the like. The recorded feedback data may be sent to the controller (124) which is also configured to process the data.

The robotic device (108) may further include one or more actuators (610) arranged to operate the robotic device and move the body (and related parts) of the robotic device. The actuators (610) may receive instructions from the controller (602).

The teleoperation device (107) may have similar components to that of the robotic device (108), shown in FIG. 6. For the sake of conciseness, these components are not shown in a separate drawing.

The invention discussed herein therefore provides a method and system for interactive data collection dataset augmentation, and iterative refinement of machine learning policies for robotic systems. Importantly, the system uses morphology-matching teleoperation devices, which are configured to closely replicate the kinematic structure of the robotic device being controlled. These morphology-matching teleoperation devices allow for seamless human intervention during both autonomous and teleoperated tasks, enabling real-time corrections to refine and improve robot learning policies.

The present invention therefore may overcome the challenges present in prior art systems by introducing a system that integrates morphology-matching teleoperation devices with bidirectional control. The morphology-matching teleoperation device enables seamless human intervention during robot operation, allowing a human operator to correct errors in real time while the robotic device executes its learned policy. This interactive, human-in-the-loop approach ensures that corrections are collected on-policy, aligning the training and test distributions. The corrected trajectories are then used to refine the behaviours of the previous learned policy using fine-tuning or reinforcement learning and this iteratively continues until the policy achieves a desired level of performance.

As discussed above, the system and method may be operated in various different scenarios / configurations.

### Configuration 1 (FIGS. 2A and 2B)

In this embodiment, the teleoperation device and robotic device are combined into one unified device.

The device is configured to alternate seamlessly between autonomous operation and teleoperation, where a human operator may provide input, such as movement corrections.

Any operator input during autonomous execution is logged, stored, and used to refine a machine learning policy executing during operation of the device.

This configuration simplifies hardware requirements and enhances usability by enabling direct human interaction with the robotic system.

### Configuration 2 (FIG. 2C)

In this embodiment there is both a teleoperation device and a robotic device provided separately. A trained machine learning policy is deployed to operate on the teleoperation device.

The robotic device is configured to mirror the teleoperation device's movements using the sensor array and controller.

A human operator may intervene to adjust the teleoperation device so as to correct deviations observed in the behaviour of any one of the robotic device or the teleoperation device.

These corrected trajectories (inputs) are recorded, aggregated into a training dataset, and used to retrain the policy executing on the teleoperation device.

This configuration enables the system to be compatible with any off-the-shelf robot for which a corresponding morphology matching teleoperation device may be created.

### Configuration 3 (FIG. 2C)

In this embodiment the robotic device and the teleoperation device are also separate devices. The robotic device is configured to execute the policy autonomously when requested.

The teleoperation device, at least partially under the control of an operator, is configured to mirrors the robotic device's movements. This mirroring is again made possible via the sensor array and controller of one or both of the teleoperation device and the robotic device.

If any inaccuracies are observed in the execution of the policy by the robotic device, the human operator may apply manual corrections via the teleoperation device. The augmented trajectories (human inputs) are recorded and used for on-policy refinement, similar to configuration 2, above.

This configuration enables the system to interface with any off-the-shelf robot for which we a corresponding morphology matching teleoperation device may be created.

The system may be configured to operate in two primary modes in all of the above configurations. These modes include a teleoperation mode and an autonomous mode. In the teleoperation mode, the teleoperation device is directly, manually controlled by a human operator for task execution or data collection. In the autonomous mode, the robotic device or the teleoperation device may autonomously (or at least semi-autonomously) be controlled by the server and its components to perform tasks/actions independently.

It should be appreciated that, in order to provide user real-time user input, the system should be able to switch between the two modes as and when required. To this end, the system may include one or more operator-controlled switches. These switches are configured to provide the operator with precise, immediate control over the operational mode and thereby allow real-time intervention.

The unified system, shown in configuration 1 above, may transitions between operational phases as follows:

### Autonomous Mode

The robotic device operates under the control of a trained machine learning policy.

The system continuously performs tasks, such as assembling parts, manipulating objects, or navigating an environment, based on the executed policy.

The sensor array records task execution data and logs any deviations from expected performance, such as encountering an edge case or obstacle.

### Operator-Initiated Intervention (Mode Switch)

When the operator detects suboptimal behaviour or a need for adjustment, they can activate the mode-switching mechanism to intervene.

For example, the operator may press a button, tap a foot pedal, or issue a voice command to toggle into teleoperation mode. The transition is immediate, ensuring no interruption in the task's progress.

Once the mode-switching mechanism is activated autonomous control is paused, and actuators of the robotic device switch to a passive mode to allow human-driven control.

The robotic system enters teleoperation mode, mirroring the operator's movements.

### Teleoperation Mode

In teleoperation mode, the operator directly manipulates the robotic device's movements using the morphology-matching device.

The sensor array and controller capture the operator's adjustments, including joint positions, applied torques, and end-effector trajectories.

For example, the operator may reposition an arm to grasp an object more effectively or adjust a gripping force to prevent damage to a fragile item.

These corrections are applied in real-time, allowing the robotic device to adapt immediately to the operator's guidance.

### Return to Autonomous Mode

Once the operator completes the intervention, they deactivate the mode-switching mechanism to resume autonomous operation.

The system transitions seamlessly back to autonomous control, using the refined trajectory as a data sample for refining the machine learning model.

This iterative process ensures that autonomous operations continue with improved performance, guided by the operator's corrections.

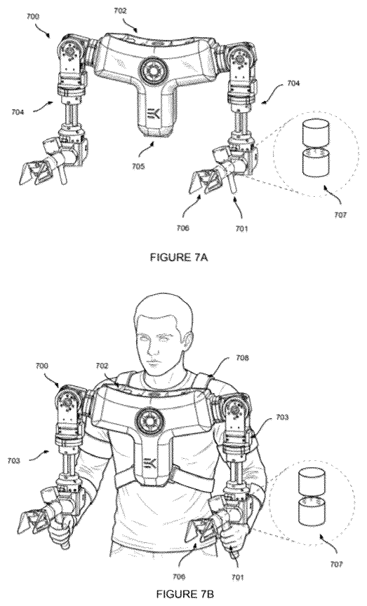

An example of a unified teleoperation device and robotic device will be described with reference to FIGS. 7A and 7B.

These figures illustrate the wearable configuration of the unified system, designed for seamless transitions between autonomous operation and human-led data collection or intervention.

The teleoperation device (700) includes a control handle (701), robot gripper (706), mechanical compliance mechanism (707), robotic arm joints (703), robotic torso (702) and a mounting point (705).

The teleoperation device shown in 7B further includes a harness system (708).

The robot gripper (706) represents the end-effector mechanism responsible for interacting with the environment. The control handle (701) is a physical interface allowing a human operator to manually guide the robotic arm during data collection or corrective intervention. The gripper (706) can include buttons to open and close the gripper as required. Alternatively, the user could directly open and close the grippers (706) with their thumb and index finger.

The mechanical compliance mechanism (707) is situated between the control handle (701) and the gripper (706). It serves as a kinematic transducer that allows for a degree of “play” or relative motion between the user's hand and the robot's end-effector.

The robotic arm (704) is a multi-link, multi-joint manipulator that facilitates robotic objectives. In this example, two robot arms (704) are shown but a person skilled in the art would appreciate that more or fewer robot arms (704) could be used depending on the desired implementation.

The robotic torso (702) is a structural chassis that connects the robotic arms (704). It is ergonomically contoured to be worn by a user during teleoperation or mounted to a support structure for autonomous tasks.

The mounting point (705) is a localised interface on the torso (702) for securing the device to a static or mobile base during autonomous operation.

The harness system (708) is a series of adjustable straps or fasteners that secure the torso (702) to the operator, ensuring that the user's shoulder and chest movements are aligned with the robot's frame of reference for intuitive control.

This mechanical compliant mechanism may include, but is not limited to, spring offsets, elastomeric dampers, or multi-axis flexures capable of capturing normal forces, torques, or a combination thereof. A person skilled in the art would appreciate that other materials could be used depending on their desired implementation.

In systems lacking dedicated force-torque sensors, rigid contact with an environment (e.g., pressing a table) results in static joint data that fails to capture the magnitude of applied force. The mechanism (707) translates these applied forces into subtle, measurable displacements of the robotic arm joints (703). By allowing this slight movement, the robot's internal encoders capture a “proprioceptive signature” of the force being applied. When training a machine learning model (e.g., via Behavioural Cloning), the model learns that a specific visual context requires not just a position, but a continued downward joint movement-effectively “learning” the force requirement through kinematic data alone.

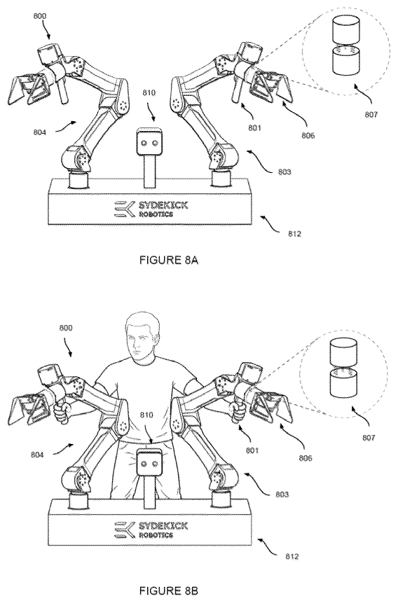

An example of a unified teleoperation device and robotic device will be described with reference to FIGS. 8A and 8B.

These figures illustrate the table-top instantiation of the unified teleoperation and robot configuration for human-in-the-loop data collection and corrective feedback.

The teleoperation device (800) includes a control handle (801), robot gripper (806), mechanical compliance mechanism (807), robotic arm joints (803), robotic torso (802), a camera (810) and a tabletop base station (812).

The robot gripper (806) represents the end-effector mechanism responsible for interacting with the environment. The control handle (801) is a physical interface allowing a human operator to manually guide the robotic arm during data collection or corrective intervention. The gripper (806) can include buttons to open and close the gripper as required. Alternatively, the user could directly open and close the grippers (806) with their thumb and index finger.

The mechanical compliance mechanism (807) is situated between the control handle (801) and the gripper (806). It serves as a kinematic transducer that allows for a degree of “play” or relative motion between the user's hand and the robot's end-effector.

The robotic arm (804) is a multi-link, multi-joint manipulator that facilitates robotic objectives. In this example, two robot arms (804) are shown but a person skilled in the art would appreciate that more or fewer robot arms (804) could be used depending on the desired implementation.

The camera (810) is a vision system for capturing image data during data collection/intervention that can be used by the machine learning model to understand the state of the environment.

The tabletop base station (812) is a mount for the robot arms (804) that can be attached to a tabletop for operation. In this example, the tabletop base station (812) acts as the housing for an onboard computer used to run the machine learning models and controls for the robot arms (804).

In systems lacking dedicated force-torque sensors, rigid contact with an environment (e.g., pressing a table) results in static joint data that fails to capture the magnitude of applied force. The mechanism (807) translates these applied forces into subtle, measurable displacements of the robotic arm joints (803). By allowing this slight movement, the robot's internal encoders capture a “proprioceptive signature” of the force being applied. When training a machine learning model (e.g., via Behavioural Cloning), the model learns that a specific visual context requires not just a position, but a continued downward joint movement-effectively “learning” the force requirement through kinematic data alone.

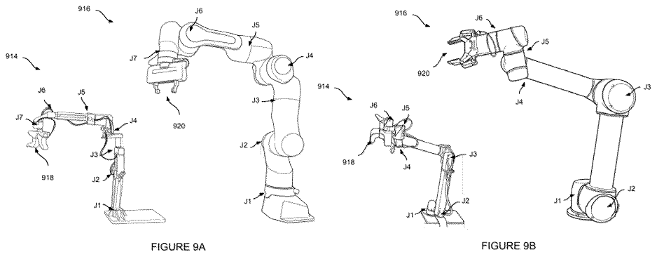

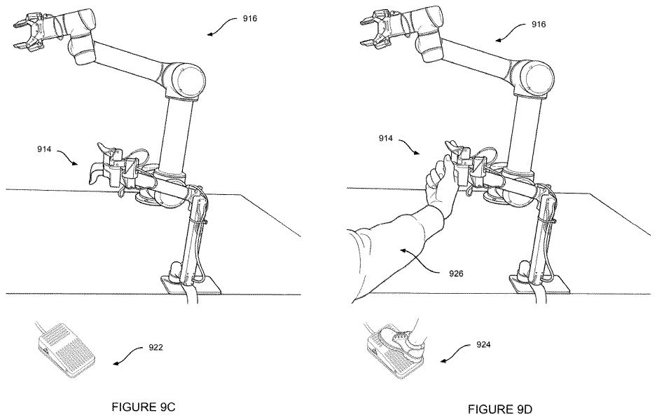

An example of a morphology matching teleoperation device for controlling a robotic device will be described with reference to FIGS. 9A to 9D.

These figures illustrate the more general “leader-follower” architecture of the system, specifically highlighting the morphology-matching teleoperation device designed for high-fidelity data collection and real-time human intervention.

FIGS. 9A and 9B demonstrate that the teleoperation device (914) is a kinematic mirror of the robot arm (916) it controls.

The morphology-matching teleoperation device (914) is a control interface that has proportional link lengths, joint offsets, and degrees of freedom (DoF) of the target robot. The follower robot arm (916) is the primary manipulator executing tasks in the environment. The operator interface (918) is a handle located at the end-effector of the teleoperation device (914) and includes a manual trigger for proportional or binary control of the robot gripper (920).

FIG. 9A further includes a 7-DoF configuration is where joints J1 to J7 on the teleoperation device correspond exactly to the joint axes of the robot. FIG. 9B further includes a 6-DoF configuration is shown (J1 to J6).

Each joint (J) on the teleoperation device (914) is equipped with both a rotational encoder for sensing human input and an actuator for haptic feedback and autonomous synchronisation.

FIGS. 9C and 9D illustrate the transition between autonomous execution and human-led correction, enabled by the bilateral synchronisation of the two devices used for collecting intervention data to improve the machine learning model. FIG. 9C shows the system when autonomous execution is operating and FIG. 9D shows the system led by human correction. Intervention triggers (992, 924) are a foot pedal, button, or similar input device allows the operator (926) to instantly override the autonomous policy

In the autonomous state, a machine learning model (e.g., a policy trained via behavioural cloning) drives the system. The model can command the teleoperation device (914) directly, with the robot (916) following its joint positions, or vice versa. This ensures that the physical joint configuration of the teleoperation device constantly mirrors the real-time state of the robot joints.

Upon activation of the trigger (924), the actuators in the teleoperation device (914) transition to a “gravity-compensated” or “passive” state. Because the device was already synchronised to the robot's joint position during autonomous mode, the operator (926) can engage with the handle seamlessly, without the “jump” in motion typically found in non-morphological teleoperation or pose-matching teleoperation devices.

The operator (926) then performs a “corrective demonstration.” The resulting data—including joint positions, velocities, and synchronised camera feeds, etc.—is captured to retrain and refine the machine learning model.

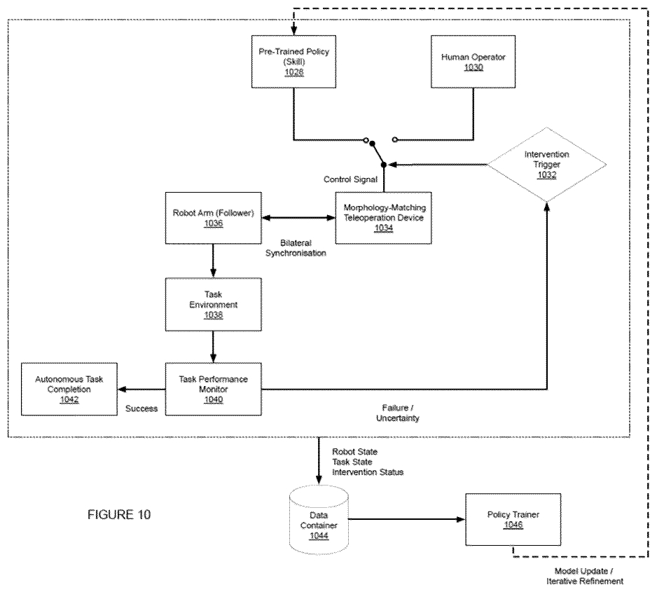

An example of a system flow architecture for iterative policy refinement using an actuated, morphology-matched interface bridge is shown in FIG. 10.

The system architecture is centred around a Control Signal Multiplexer (Switch) that manages the flow of authority to the hardware.

The pre-trained policy (skill) (1028) is the primary control source during autonomous operation. It outputs a policy-driven control signal based on real-time task observations. The operator (1030) is the secondary control source, where the operator (1030) provides manual control signals through the physical interface of teleoperation device (1034).

An intervention trigger (1032) is a physical input (e.g., a foot pedal) that acts as the selector for the multiplexer. In a default state, the switch routes the signal from the Policy (1028) to the hardware. Upon activation, the switch toggles to route the signal from the operator (1030), granting immediate manual authority.

The morphology-matching teleoperation device (1034) serves as the physical state bridge between the controllers and the robot. Unlike passive controllers, the device (1034) is actively driven by the autonomous policy (1028) while in autonomous mode. This ensures that the device's joints are always aligned with the robot's current pose.

The follower robot arm (1036) is the physical agent executing the task. It maintains bilateral synchronisation with the teleoperation device (1034), ensuring that any movement in one is immediately reflected in the other, regardless of control. A person skilled in the art would appreciate that if the bilaterial synchronisation may not be required if the robotic device (1034) is a unified system as described in reference to FIGS. 7A and 7B.

The task environment (1038) is the physical space where the robot arm (1036) performs work. The task performance monitor (1040) is a logic block that evaluates the robot's progress.

If the task reaches autonomous task completion (1042) without interference, the trial is logged as a success. If a state of failure or uncertainty is detected (either by the human or the monitor), the intervention trigger (1032) is engaged to switch control modes.

To facilitate the capture of all relevant parameters and monitoring of subsystems, the system captures a continuous stream of data relating to the robot state, task state and Intervention status.

A data container (1044) acts a centralised storage module to partition nominal, successful, data from corrective (intervention) date.

The data containers (1044) store various task and robot relevant information throughout the process, including but not limited to: robot joint positions, robot joint velocities, teleoperation device joint positions, teleoperation device joint velocities, sensor information (images, tactile, depth etc). A person skilled in the art would appreciate that other types of data could be stored depending on the desired implementation.

A policy trainer (1046) acts as a machine learning engine that uses the partitioned data to update the model. By focusing on the high-value intervention data, the trainer can effectively resolve distribution shifts.

The updated model is subsequently fed back to the pre-trained policy (1028) section to create a closed-loop system where the robot's performance improves through successive cycles of autonomous execution and human correction.

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Throughout the specification and claims unless the context requires otherwise the word ‘comprise’ or variations such as ‘comprises’ or ‘comprising’ will be understood to imply the inclusion of a stated integer or group of integers but not the exclusion of any other integer or group of integers.

1. A teleoperation device for use in collecting data for fine-tuning a machine learning model to control a robotic device, the teleoperation device comprising:
a) a morphology matching kinematic chain structure corresponding to a kinematic chain structure of the robotic device, wherein each joint and link of the robotic device has a corresponding joint and link on the teleoperation device;
b) actuators configured to enable movement of the teleoperation device joints under autonomous control of a machine learning model or under operator input;
c) control circuitry configured to synchronise movement of the teleoperation device with movement of the robotic device such that movements of one device are mirrored by the other; and
d) a data interface configured to transmit operator input data and corrective behaviours to a server for aggregation into a training dataset associated with the machine learning model.

2. A teleoperation device according to claim 1, wherein the teleoperation device has a kinematic structure corresponding to a kinematic structure of the robotic device so that the teleoperation device mirrors joint actions performed by the robotic device.

3. A teleoperation device according to claim 1, wherein the teleoperation device includes a number of joints equal to or more than the number of joints in the robotic device.

4. A teleoperation device according to claim 3, wherein the teleoperation device includes a number of joints more than the number of joints in the robotic device.

5. A teleoperation device according to claim 4, wherein a mapping function is trained to map the joint movements of the teleoperation device is matched to the joint movements of the robotic device by at least one of:
a) imitation learning;
b) reinforcement learning; and
c) supervised learning.

6. A teleoperation device according to claim 5, wherein the matching is performed by at least one of the data interface and the server.

7. A teleoperation device according to claim 1, wherein the teleoperation device is configured to enable operator intervention to provide corrective behaviour that is collected as feedback data.

8. A teleoperation device according to claim 1, wherein the teleoperation device and the robotic device comprise a single unified device.

9. A teleoperation device according to claim 1, wherein the teleoperation device is configured to support passive and active control modes, wherein:
a) in the passive mode, an operator of the teleoperation device may directly drive the joints of the teleoperation device while the actuators move freely; and
b) in the active mode, human input forces are detected and an impedance-based controller applies velocity or torque commands to the actuators.

10. A teleoperation device according to claim 9, wherein the teleoperation device includes bidirectional actuators and the bidirectional actuators are configured to enable passive control and active control of the robotic device.

11. A teleoperation device according to claim 9, wherein, once the operator completes the intervention, autonomous operation is resume using the refined trajectory as the new data sample that can be stored for improving the machine learning model.

12. A method for collecting data for fine-tuning a machine learning model to control a robotic device, the method comprising the steps of:
a) receiving a request for initiating an action to be performed by a robotic device;
b) processing the request to identify a machine learning model associated with the requested action;
c) providing access to the machine learning model to a controller of one or both of the robotic device a teleoperation device and instructing the controller of the relevant device to execute the model, wherein the teleoperation device includes a morphology matching kinematic chain structure corresponding to a kinematic chain structure of the robotic device, wherein each joint and link of the robotic device has a corresponding joint and link on the teleoperation device;
d) receiving feedback data from at least one of the controllers, the feedback data including data relating to a human-based input provided via the teleoperation device; and
e) processing the feedback data to update the machine learning model, wherein processing the feedback data includes:
(i) aggregating a training dataset associated with the machine learning model with the feedback data; and
(ii) re-training the machine learning model using the aggregated training dataset.

13. A method according to claim 12, wherein processing the request includes extracting a user identifier and an action identifier from the action request.

14. A method according to claim 13, wherein at least one of:
a) the user identifier is associated with a user platform and/or user device and the action identifier is associated with one or more machine learning models; and
b) determining whether a user associated with the user identifier has access to the one or more machine learning models associated with the action identifier.

15. A method according to claim 12, wherein the feedback data includes pre-processed data logs based on data captured by a sensor array of the robotic device and/or a sensor array of the teleoperation device.

16. A method according to claim 15, the feedback data further includes pre-processed data logs based on data captured by a user device.

17. A method according to claim 12, wherein determining a performance score of the model executed by the controller.

18. A method according to claim 17, wherein at least one of:
a) determining the performance score includes:
(i) processing the feedback data to identify data logs associated with actions performed by the robotic device;
(ii) matching the actions with a predefined success criterion to determine a ratio of successful actions to attempted actions; and
(iii) determining a performance score based on the ratio;
b) the success criterion is based on whether the model succeeds to solve a task in a real-world or simulation environment; and
c) storing the machine learning model as a skill if the performance score is greater than a threshold.

19. A method according to claim 12, wherein at least one of:
a) providing access to the machine learning model includes transmitting the model data to the controller or transmitting a pointer to model data stored in a repository maintained by the server; and
b) further comprising:
(i) selecting a skill or policy from a skill library of previously trained skills;
(ii) executing the skill using state information as input to generate joint actions of the robotic device;
(iii) enabling corrective input via the teleoperation device to correct erroneous actions; and
(iv) recording corrected trajectories for inclusion in the training dataset.

20. A system for collecting data for fine-tuning a machine learning model to control a robotic device, the system comprising:
a) a memory for storing computer-readable program code and a processor for executing the computer-readable program code;
b) a request receiving component configured to receive a request for initiating an action to be performed by a robotic device;
c) a request processing component configured to process the request to identify a machine learning model associated with the requested action;
d) a machine learning component configured to provide access to the machine learning model to a controller of one or both of the robotic device and a teleoperation device and instructing the controller to execute the model;
e) a feedback receiving component configured to receive feedback data from at least one of the controllers, the feedback data including data relating to a human-based input provided via the teleoperation device; and
f) a feedback processing component configured to process the feedback data to update the machine learning model, wherein processing the feedback data includes aggregating a training dataset associated with the machine learning model with the feedback data and re-training the machine learning model using the aggregated training dataset.
